(19) United States
(12) Patent Application Publication
(10) Pub. No.: US 2023/0172250 A1
(43) Pub. Date: Jun. 8, 2023

(54) ANTI-HAIR LOSS SOLID BEVERAGE AND PREPARATION METHOD THEREOF

(71) Applicant: Meyer Health Jiangsu Co., Ltd., Taizhou (CN)

(72) Inventors: Hanping Shi, Taizhou (CN); Ru Jia, Taizhou (CN)

(21) Appl. No.: 17/662,073

(22) Filed: May 4, 2022

Related U.S. Application Data
(63) Continuation of application No. 17/540,243, filed on Dec. 2, 2021.

(51) Int. Cl. A23L 33/18; A23L 33/125; A61Q 7/00; A23P 10/20; A61K 8/64

(52) U.S. Cl. CPC A23L 33/18; A23L 33/125; A23P 10/20; A61K 8/64; A61Q 7/00

(57) ABSTRACT

The present disclosure relates to the field of functional foods, and particularly relates to an anti-hair loss solid beverage and a preparation method thereof. The anti-hair loss solid beverage includes the following raw materials in percentage by mass: 4% to 6% of lytodark, 35% to 48% of non-dairy creamer, 5.5% to 7% of vegetable fat powder, 40% to 54% of maltodextrin, 0.5% to 1 % of sucralose, and 0.5% to 1.5% of essence. The lytodark is prepared from nucleoprotein peptide, coffee fruit extract, and pumpkin seed oil.

### CROSS-REFERENCE TO RELATED APPLICATIONS

This application is a continuation of U.S. Pat. Application No. 17/540,243, filed on Dec. 2, 2021, entitled “ANTI-HAIR LOSS SOLID BEVERAGE AND PREPARATION METHOD THEREOF”, which is hereby incorporated by reference.

### TECHNICAL FIELD

The present disclosure relates to the field of functional foods, and particularly relates to an anti-hair loss solid beverage and a preparation method thereof.

### BACKGROUND

As a part of the scalp of a human body, the hair plays an important role in protecting the head while maintaining the personal image. Normal persons have about 10 ten thousand to 12 ten thousand hairs, each of which has a complete hair growth cycle including a growth period, a catagen period, and a final alopecia period, and have about 100 hairs loss per day with a substantially equivalent number of new hairs being produced, thereby completing normal metabolism.

Alopecia is divided into physiological alopecia and pathological alopecia. Physiological alopecia is a normal physiological phenomenon caused by the above-mentioned metabolism of the human body, and pathological alopecia is abnormal hair loss, so that the quantity of the alopecia is more than the quantity of new hair growth. Pathological alopecia is generally classified into: seborrheic alopecia; female postpartum alopecia, climacteric alopecia and other endocrine alopecia; alopecia caused by nutritional metabolism problems such as weight loss, vitamin deficiency, malnutrition and the like; alopecia caused by radiotherapy, chemotherapy, and folliculitis; alopecia caused by chemical stimulation such as improper selection of washing and caring products, perm, hair dyeing and the like; hair thinning, withered and yellow, thin, soft, easy breakage, unhealthy hair and the like caused by great mental stress. In a process of growth of human hair and hair follicles, two major enzymes play a key role: tyrosinase and 5-α reductase.

Tyrosinase, also known as polyphenol oxidase, catechol oxidase, aged cheese enzyme, etc., is a copper-containing oxidoreductase with a complex structure and containing multiple subunits, and widely exists in microorganisms, animals, plants, and human bodies. Tyrosinase has a variety of characteristic catalytic activities, which can be used as both tyrosine hydroxylase and dopa oxidase, as well as 5, 6-dihydroxyindole oxidase, and also can be used as a rate-limiting enzyme for regulating melanin, and plays a crucial role in the melanin generation process. Abnormal expression of tyrosinase can directly or indirectly lead to human diseases such as malignant melanoma, skin spots, vitiligo, alopecia, etc. Therefore, activation of the tyrosinase activity can promote the generation of hair melanin while stimulating the growth of hair follicles, thereby achieving the purposes of blacking hair and growing hair. At present, there are many researches and related inventions on substances for inhibiting tyrosinase, but few researches on substances for activating tyrosinase, and more representative are some tyrosinase activating substances selected from medicinal materials, such as fructus psoraleae, radix puerariae, radix salviae miltiorrhizae and the like. These medicinal materials are relatively expensive, and the extraction and purification processes are complex, so that the search for a tyrosinase activating substance which is cheap and easy to obtain and has a simple process is urgent.

The 5-α reductase mainly has the functions of catalyzing testosterone to be converted into dihydrotestosterone, and the dihydrotestosterone can be combined with androgen receptors in target cells of human hair follicles to inhibit the normal growth and development of hair papillae, disturb the normal metabolism of hair follicle stem cells, enable hairs to enter the alopecia period in advance and cause alopecia symptoms. Therefore, the inhibition of the production of dihydrotestosterone is also a key point for treating alopecia, which depends on the inhibition of the activity of 5-α reductase. A type of chemical inhibitors are at most currently used, such as finasteride which is a specific type II 5-α reductase inhibitor, can inhibit the conversion of testosterone in blood serum into dihydrotestosterone after administration, and has a remarkable promoting effect on hair growth. Other commercially available hair growth promoting agents also include drugs such as carpronium chloride and minoxidil, which act as hormones that inhibit the conversion of testosterone into dihydrotestosterone. However, the testosterone inhibitors and testosterone hormones mentioned above often cause side effects of inhibiting male sexual function in use, resulting in adverse reactions such as low libido, erectile dysfunction, decreased ejaculatory amount, ejaculatory disorder, skin rash, breast enlargement or tenderness, and further, these hair growth pharmaceutical preparations may cause skin abnormalities when applied to alopecia.

At present, there are few kinds of products that can effectively treat the alopecia in the market, and most of the products are hormone medicines or chemical preparations. Although the products have certain treatment effects, most of the products can treat the symptoms and the root causes but not the root causes, the medicines or the preparations can cause harm to the body after long-term use, and the medicines or the preparations have the defects of coarse mouthfeel, poor palatability, low bioavailability and the like. However, with the development of modern medicine and the improvement and change of the public consumption concept and health consciousness, the priority of medication treatment is lower and lower, and people are more eager to treat and prevent the diseases by regulating the diet and utilizing healthy diets that regulate the metabolism of human bodies, supplement nutrition, and the like. Therefore, there is an urgent need to research and develop a functional beverage product which can treat both symptoms and root causes, has the advantages of anti-hair loss, good taste, and high bioavailability to supply market.

### BRIEF DESCRIPTION OF THE DRAWINGS

In order to more clearly illustrate the technical solutions in this embodiment disclosure, the drawings used in the embodiments or the description of the prior art will be briefly introduced below. It should be understood that, the drawings in the following description are only examples of the present disclosure. For those skilled in the art, other drawings can be obtained based on these drawings without creative works.

FIG. 1 is a flow chart of a preparation method according to an embodiment of the present disclosure.

### DETAILED DESCRIPTION

In order to make the objectives, technical solutions, and advantages of the present disclosure clearer, the present disclosure will be further described in detail below with reference to the embodiments. It should be understood that, the embodiments described herein are only for explaining the present disclosure and are not intended to limit the present disclosure.

Embodiments of the present disclosure provide an anti-hair loss solid beverage, comprising the following raw materials in percentage by mass: 4% to 6% of lytodark, 35% to 48% of non-dairy creamer, 5.5% to 7% of vegetable fat powder, 40% to 54% of maltodextrin, 0.5% to 1% of sucralose, and 0.5% to 1.5% of essence. The lytodark is prepared from the following components: 25% to 35% of nucleoprotein peptide, 36% to 44% of coffee fruit extract, and 26% to 38% of pumpkin seed oil.

The nucleoprotein peptide is prepared by enzymolysis, purification, and drying of deep-sea fish milt. The coffee fruit extract is prepared by extracting with ethanol aqueous solution, concentrating, and drying the coffee fruit.

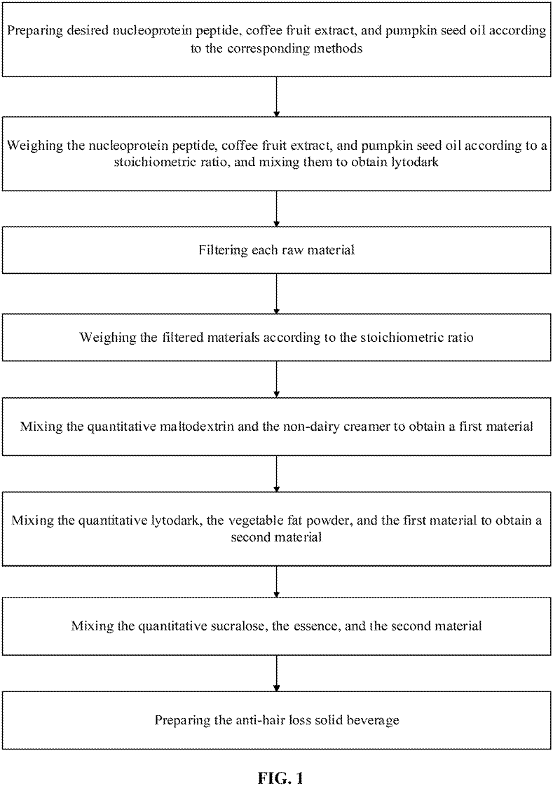

The present disclosure also provides a method of preparing an anti-hair loss solid beverage, which illustratively, as shown in FIG. 1, includes the following steps.

At preparation of nucleoprotein peptide, deep-sea fish milt is cleaned, cut into pieces, and freeze-dried for later use. The treated milt is soaked in a sodium chloride solution with a mass fraction of 8% to 15% for 1 h to 2 h, then filtered, and a filter cake is washed with deionized water and repeated for five times. An appropriate amount of distilled water is added into the soaked and filtered milt, fully stirred, and then nucleoprotease is added for extraction for 2 h to 4 h at a temperature of 40° C. to 60° C., wherein the nucleoprotease has a dosage of 3500 U/g to 5500 U/g, and a material-liquid ratio is 1: (5-10). The extracting solution is heated to 80° C. to 100° C. to inactivate the nucleoprotease, and centrifuged for 20 min at a temperature of 25° C. at 5000 rpm to obtain a supernatant purified solution. The purified solution is dried at 30° C. to 40° C., and then freeze-dried to obtain the desired nucleoprotein peptide.

At preparation of coffee fruit extract, coffee beans are removed from a fresh coffee cherry to obtain a coffee fruit. After the coffee fruit is ground into powder, the powder is dissolved in an ethanol aqueous solution with a mass fraction of 25% for extraction, wherein the extraction time is 0.5 h to 2 h, the temperature is 60° C. to 70° C., and the ethanol aqueous solution has a dosage of 10 mL/g to 20 mL/g. The extracting solution is concentrated under reduced pressure to remove the solvent, and finally is dried at 50° C. to 60° C. to obtain the desired coffee fruit extract.

At preparation of pumpkin seed oil, fresh pumpkin seeds are ground into powder. Distilled water with a weight 5-8 times that of the pumpkin seed powder is added into pumpkin seed powder, and fully stirred. Then, cellulase with a mass fraction of 0.5% and pectinase with a mass fraction of 0.1% are added, and enzymolysis is carried out for 3 h to 5 h at a temperature of 25° C.: to 40° C. The enzymatic hydrolysate is heated to 80° C. to 90° C. to inactivate the enzyme, and then centrifuged for 20 min at 5000 rpm and 25° C. to obtain an upper-layer free liquid. The upper-layer free liquid is concentrated under reduced pressure, and finally dried at 30° C. to 40° C. to obtain the desired pumpkin seed oil.

The prepared nucleoprotein peptide, coffee fruit extract, and pumpkin seed oil are quantitatively taken according to a mass ratio of 25% to 35% of nucleoprotein peptide, 36% to 44% of coffee fruit extract, and 26% to 38% of pumpkin seed oil.

The quantitative nucleoprotein peptide, coffee fruit extract, and pumpkin seed oil are mixed to obtain lytodark.

The lytodark, non-dairy creamer, vegetable fat powder, maltodextrin, sucralose, and essence are filtered, respectively.

The raw materials are quantitatively taken according to a mass ratio of 4% to 6% of lytodark, 35% to 48% of non-dairy creamer, 5.5% to 7% of vegetable fat powder, 40% to 54% of maltodextrin, 0.5% to 1% of sucralose, and 0.5% to 1.5% of essence.

The quantitative maltodextrin and non-dairy creamer are mixed to obtain a first material.

The quantitative lytodark, the vegetable fat powder, and the first material are mixed to obtain a second material.

The quantitative sucralose, the essence, and the second material are mixed.

The anti-hair loss solid beverage is prepared.

Specifically, the filtering includes filtering lytodark, non-dairy creamer, vegetable fat powder, maltodextrin, sucralose, and essence by using a sieve, respectively.

The sieve has a mesh number of 40 meshes or 60 meshes.

The purity of each of the raw materials is increased by filtration, and the powder is finer, so that the mixing effect is better.

Specifically, the mixing the quantitative maltodextrin and the non-dairy creamer includes:
adding maltodextrin and non-dairy creamer into a stirrer, and stirring uniformly for a first time in a sealed state;
the stirring for the first time has a speed of 20 r/min to 40 r/min.

Specifically, the mixing the quantitative lytodark, the vegetable fat powder, and the first material includes:
adding lytodark and vegetable fat powder into a stirrer containing the first material, and stirring uniformly for a second time in a closed state;
the stirring for the second time has a speed of 20 r/min to 40 r/min.

Specifically, the mixing the quantitative sucralose, the essence, and the second material includes:
adding the sucralose and the essence into a stirrer containing the second material, and stirring uniformly for a third time in a closed state until no agglomerate is present;
the stirring for the third time has a speed of 20 r/min to 40 r/min.

### Example 1

In Example 1, a preparation of 100 g of an anti-hair loss solid beverage was taken as an example.

Preparation of nucleoprotein peptide: deep-sea fish milt was cleaned, cut into pieces, and freeze-dried for later use. The treated milt was soaked in a sodium chloride solution with a mass fraction of 8% to 15% for 1 h to 2 h, then filtered, and a filter cake was washed with deionized water and repeated for five times. An appropriate amount of distilled water was added into the soaked and filtered milt, fully stirred, and then nucleoprotease was added for extraction for 2 h to 4 h at a temperature of 40° C. to 60° C., wherein the nucleoprotease had a dosage of 3500 U/g to 5500 U/g, and a material-liquid ratio was 1: (5-10). The extracting solution was heated to 80° C. to 100° C. to inactivate the nucleoprotease, and centrifuged for 20 min at a temperature of 25° C. at 5000 rpm to obtain a supernatant purified solution. The purified solution was dried at 30° C. to 40° C., and then freeze-dried to obtain the desired nucleoprotein peptide.

Preparation of coffee fruit extract: coffee beans were removed from a fresh coffee cherry to obtain a coffee fruit. After the coffee fruit was ground into powder, the powder was dissolved in an ethanol aqueous solution with a mass fraction of 25% for extraction, wherein the extraction time was 0.5 h to 2 h, the temperature was 60° C. to 70° C., and the ethanol aqueous solution had a dosage of 10 mL/g to 20 mL/g. The extracting solution was concentrated under reduced pressure to remove the solvent, and finally was dried at 50° C. to 60° C. to obtain the desired coffee fruit extract.

Preparation of pumpkin seed oil: fresh pumpkin seeds were ground into powder. Distilled water with a weight 5-8 times that of the pumpkin seed powder was added into pumpkin seed powder, and fully stirred. Then, cellulase with a mass fraction of 0.5% and pectinase with a mass fraction of 0.1% were added, and enzymolysis was carried out for 3 h to 5 h at a temperature of 25° C. to 40° C. The enzymatic hydrolysate was heated to 80° C. to 90° C. to inactivate the enzyme, and then centrifuged for 20 min at 5000 rpm and 25° C. to obtain an upper-layer free liquid. The upper-layer free liquid was concentrated under reduced pressure, and finally dried at 30° C. to 40° C. to obtain the desired pumpkin seed oil.

The prepared nucleoprotein peptide, coffee fruit extract, and pumpkin seed oil were quantitatively taken according to a mass ratio of 26% of nucleoprotein peptide, 36% of coffee fruit extract, and 28% of pumpkin seed oil.

2.6 g of nucleoprotein peptide, 3.6 g of coffee fruit extract, and 3.8 g of pumpkin seed oil were mixed to obtain 10 g of lytodark.

An anti-hair loss solid beverage was prepared by using 4% of lytodark, 35% of non-dairy creamer, 5.5°,% of vegetable fat powder, 54% of maltodextrin, 0.5% of sucralose, and 1% of essence.

The lytodark, non-dairy creamer, nutrient components, vegetable fat powder, maltodextrin, sucralose, and essence were filtered by using a sieve, respectively. The sieve had a mesh number of 40 meshes.

54 g of maltodextrin and 35 g of non-dairy creamer were added into a stirrer, and uniformly stirred at 20 r/min in a sealed state to obtain a first material.

4 g of lytodark and 5.5 g of vegetable fat powder were added into a stirrer containing the first material, and uniformly stirred at a stirring speed of 20 r/min in a closed state to obtain a second material.

0.5 g of sucralose and 1 g of essence were added into a stirrer containing the second material, stirred uniformly at a stirring speed of 20 r/min in a closed state, and discharged after checking that no agglomerate was present, thereby preparing the anti-hair loss solid beverage.

### Example 2

In Example 2, a preparation of 100 g of an anti-hair loss solid beverage was taken as an example.

The preparation processes of the nucleoprotein peptide, the coffee fruit extract, and the pumpkin seed oil were the same as those in Example 1. The prepared nucleoprotein peptide, the coffee fruit extract, and the pumpkin seed oil were quantitatively taken according to a mass ratio of 35% of nucleoprotein peptide, 39% of coffee fruit extract, and 26% of pumpkin seed oil.

3.5 g of nucleoprotein peptide, 3.9 g of coffee fruit extract, and 2.6 g of pumpkin seed oil were mixed to obtain 10 g of lytodark.

An anti-hair loss solid beverage was prepared by using 6% of lytodark, 35% of non-dairy creamer, 7% of vegetable fat powder, 51% of maltodextrin, 0.5% of sucralose, and 0.5% of essence.

The lytodark, non-dairy creamer, vegetable fat powder, maltodextrin, sucralose, and essence were filtered by using a sieve, respectively. The sieve had a mesh number of 60 meshes.

51 g of maltodextrin and 35 g of non-dairy creamer were added into a stirrer, and uniformly stirred at 20 r/min in a sealed state to obtain a first material.

6 g of lytodark and 7 g of vegetable fat powder were added into a stirrer containing the first material, and uniformly stirred at a stirring speed of 20 r/min in a closed state to obtain a second material.

0.5 g of sucralose and 0.5 g of essence were added into a stirrer containing the second material, stirred uniformly at a stirring speed of 20 r/min in a closed state, and discharged after checking that no agglomerate was present, thereby preparing the anti-hair loss solid beverage.

### Example 3

In Example 3, a preparation of 100 g of an anti-hair loss solid beverage was taken as an example.

The preparation processes of the nucleoprotein peptide, the coffee fruit extract, and the pumpkin seed oil were the same as those in Example 1. The prepared nucleoprotein peptide, the coffee fruit extract, and the pumpkin seed oil were quantitatively taken according to a mass ratio of 25% of nucleoprotein peptide, 44% of coffee fruit extract, and 31% of pumpkin seed oil.

2.5 g of nucleoprotein peptide, 4.4 g of coffee fruit extract, and 3.1 g of pumpkin seed oil were mixed to obtain 10 g of lytodark.

An anti-hair loss solid beverage was prepared by using 4% of lytodark, 48% of non-dairy creamer, 5.5% of vegetable fat powder, 40% of maltodextrin, 1% of sucralose, and 1.5′% of essence.

The lytodark, non-dairy creamer, vegetable fat powder, maltodextrin, sucralose, and essence were filtered by using a sieve, respectively. The sieve had a mesh number of 60 meshes.

40 g of maltodextrin and 48 g of non-dairy creamer were added into a stirrer, and uniformly stirred at 40 r/min in a sealed state to obtain a first material.

4 g of lytodark and 5.5 g of vegetable fat powder were added into a stirrer containing the first material, and uniformly stirred at a stirring speed of 40 r/min in a closed state to obtain a second material.

1 g of sucralose and 1.5 g of essence were added into a stirrer containing the second material, stirred uniformly at a stirring speed of 40 r/min in a closed state, and discharged after checking that no agglomerate was present, thereby preparing the anti-hair loss solid beverage.

### Experimental Example

### Experimental Example 1: Tyrosinase Activation Test

Test Method:
Test Sample Groups:
   Group A: Ethanol extract of Example 1;
   Group B: Ethanol extract of nucleoprotein peptide;
   Group C: Ethanol extract of coffee fruit extract;
   Group D: Ethanol extract of pumpkin seed oil;
   Group E: Ethanol extract of Example 1 with nucleoprotein peptide removed;
   Group F: Ethanol extract of Example 1 with coffee fruit extract removed;
   Group G: Ethanol extract of Example 1 with pumpkin seed oil removed; and
   Blank group: ethanol

Each sample group to be tested was prepared into a sample solution to be tested with a concentration of 10 mg/mL by using phosphate buffer solution (PBS) (0.1 mmol/L, pH6.8). The sample solution to be tested was uniformly mixed with 220 ug/mL of tyrosinase, reacted at 37° C. for 10 min. Then, 0.5 mg/mL of levodopa (reaction substrate) was added, further reacted at 37° C. for 20 min. The absorbance value (A) was measured at a wavelength of 475 nm.

Tyrosinase activation rate (%) = (sample A₄₇₅ₙₘ/ blank control A₄₇₅ₙₘ -1) × 100%, and the results were shown in table 1.

TABLE 1

Tyrosinase Activation Test

| | Group A | Group B | Group C | Group D | Group E | Group F | Group G | Blank group |
|---|---|---|---|---|---|---|---|---|
| Tyrosinase Activation Rate (%) | 65.37 | 30.01 | 35.23 | 2.57 | 35.88 | 33.79 | 41.68 | N/A |

It could be seen from the data in Table 1 that the nucleoprotein peptide and the coffee fruit extract in the main ingredients of the solid beverage composition according to the present disclosure had certain promoting effects on the tyrosinase activation, and the activation rate could reach 30.01% and 35.23%, respectively, while the pumpkin seed oil hardly had promotion effects on the tyrosinase activation, and the activation rate was only 2.57%. However, when the single components were used in combination, the tyrosinase activation rate of the composition could be improved compared with those of the single components, indicating that the components had a synergistic effect on the tyrosinase activation when used in combination, and when the three single components were used simultaneously, the synergistic effect was the strongest, and the tyrosinase activation rate could reach 65.37%.

### Experimental Example 2: 5-α Reductase Inhibition Test

Test Method:
Test Sample Groups:
   Group A: Ethanol extract of Example 1;
   Group B: Ethanol extract of nucleoprotein peptide;
   Group C: Ethanol extract of coffee fruit extract;
   Group D: Ethanol extract of pumpkin seed oil;
   Group E: Ethanol extract of Example 1 with nucleoprotein peptide removed;
   Group F: Ethanol extract of Example 1 with coffee fruit extract removed;
   Group G: Ethanol extract of Example 1 with pumpkin seed oil removed; and
   Blank group: ethanol

Preparation of 5-α reductase crude enzyme solution: a healthy rat was taken to be sacrificed, and then the epididymis was taken, rinsed by using normal saline, cut into pieces, and 5-α reductase crude enzyme was extracted by using a tissue homogenate method. The components of the homogenate were 1 mmol/L of phenylmethylsulfonyl fluoride, I mmol/L of dithiothreitol, 10% of glycerol, and Imol/L of Tris-HCl buffer solution, and the pH of the homogenate was 5. A dosage ratio of the homogenate to the epididymal tissue was 5 ml: 1 g, and the homogenate was carried out for 20 min. After completion of the homogenate, centrifugation was carried out at 12000 rpm and at 4° C. for 20 min, and the supernatant was taken, namely the 5-α reductase crude enzyme solution.

300 µL of diluted 5-α reductase crude enzyme solution was added into Tris-HCI buffer solution (with the pH value of 5), 50 µL of 1 mmol/L testosterone solution was added, and then a sample group to be tested was added, and finally 50 µL of 2 mmol/L NADPII was added. The mixture was mixed uniformly, reacted at 37° C. for 1 h, then 2 mL of methanol was added, and the supernatant was taken by centrifugation at 12000 rpm. After filtration through a 22 µm filter membrane, the testosterone content in the sample to be tested was analyzed using high performance liquid chromatography. A standard curve was drawn by using a testosterone standard substance, the testosterone content in the sample was calculated according to the standard curve, and the inhibition rate of the test sample to the 5-α reductase was calculated by a calculation method as follows: inhibition rate (%) = (change in testosterone in blank control - change in sample testosterone) / change in testosterone in blank control × 100%. The results of the 5-α reductase inhibition rate test were shown in Table 2.

TABLE 2

5-α Reductase Inhibition Test

| | Group | Group | Group | Group | Group | Group | Group | Blank |
|---|---|---|---|---|---|---|---|---|
| Mean Change in Testosterone (10⁻⁵ mmol) | 0.541 | 4.624 | 4.776 | 1.201 | 0.988 | 1.095 | 4.532 | 4.856 |
| Inhibition Rate of 5-α Reductase (%) | 88.86 | 4.78 | 1.65 | 75.27 | 78.65 | 77.45 | 6.67 | N/A |

It could be seen from the data in Table 2 that the pumpkin seed oil in the main ingredients of the solid beverage composition according to the present disclosure had a certain inhibition effect on the activity of 5-α reductase, the inhibition rate could reach 75.27%, while the nucleoprotein peptide and coffee fruit extract had no obvious inhibition effect on the activity of 5-α reductase, and the inhibition rates were only 4.78% and 1.65%. However, when the single components were used in combination, the 5-α reductase inhibition rate of the composition could be improved compared with those of the single components, indicating that the components had a synergistic effect on the 5-α reductase inhibition when used in combination, and when the three single components were used simultaneously, the synergistic effect was the strongest, and the inhibition rate on the 5-α reductase could reach 88.86%.

It can be seen from the data in Table 1 and Table 2 that the solid beverage composition according to the present disclosure has a good activation effect on tyrosinase and a remarkable inhibition effect on 5-α reductase. In addition, compared with a single component, the solid beverage composition has an obvious synergistic effect when multiple components are mixed for use.

### Experimental Example 3

Animal experiments
Animals: healthy male rats;
Test Sample Groups:
   Group Example 1: Anti-hair loss solid beverage composition prepared in Example 1;
   Group Example 2: Anti-hair loss solid beverage composition prepared in Example 2;
   Group Example 3: Anti-hair loss solid beverage composition prepared in Example 3;
   Group Comparative Example 1: Anti-hair loss solid beverage composition prepared in Example 1 removing nucleoprotein peptide;
   Group Comparative Example 2: Anti-hair loss solid beverage composition prepared in Example 1 removing coffee fruit extract;
   Group Comparative Example 3: Anti-hair loss solid beverage composition prepared in Example 1 removing pumpkin seed oil; and
   Group Blank Example: Normal saline;
Test Method:
   70 healthy male rats were selected, randomly divided into 7 groups, and each group had 10 rats: group blank example, group example 1, group example 2, group example 3, group comparative example 1, group comparative example 2, and group comparative example 3.

The rats in each group were administered intragastrically with the same amount of test sample. After 7 days of adaptive feeding, the test samples were dosed 1 time/day per 1 g/kg for 3 months to observe the hair growth trend of rats, so as to determine the hair growth length and observe the number of hair follicles at the 30th day. The hair length was measured using a vernier caliper, and after administration of anesthesia to rats, the number of hair follicles was observed under a 100-fold field microscope and recorded in Table 3.

TABLE 3

Hair length and number of hair follicles in rats

| | Hair length (mm) | | | Number of follicles (Roots) |
|---|---|---|---|---|
| | One month | Two months | Three months | |
| Group Blank Example | 8.32±0.23 | 16.11±0.31 | 27.04±0.17 | 12.25±0.28 |
| Group | 13.25±0.25 | 28.51±0.38 | 39.76±0.27 | 18.89±0.31 |
| Example 1 | | | | |
| Group Example 2 | 13.54±0.19 | 29.34-+0.61 | 41.25±0.33 | 19.11±0.71 |
| Group Example 3 | 13.31±0.36 | 28.79±0.37 | 40.01±0.15 | 18.92±0.21 |
| Group Comparative Example 1 | 10.28±0.48 | 18.84±0.54 | 28.36±0.29 | 14.02±0.17 |
| Group Comparative Example 2 | 10.23±0.76 | 18.95±0.77 | 28.41±0.18 | 14.51±0.26 |
| Group Comparative Example 3 | 10.49±0.13 | 19.03±0.55 | 28.75±0.38 | 14.67±0.35 |

As can be seen from the data in Table 3, the solid beverage composition according to the present disclosure can effectively promote hair growth and increase the number of hair follicles, and the solid beverage composition having multi-components is more effective than the solid beverage having a single component.

### Experiment Example 4

### Human Clinical Trial

40 alopecia volunteers were selected. 10 volunteers ate solid beverages prepared by the formulation of Example 1, 10 volunteers ate solid beverages prepared by the formulation of Example 2, 10 volunteers ate solid beverages prepared by the formulation of Example 3, and the last 10 volunteers ate commercially available anti-hair loss solid beverages, continuously for three months, and the final test results were recorded in table 4.

TABLE 4

Human clinical trial

| | No effect | Slight effect | Significant effect |
|---|---|---|---|
| Commercially available solid beverage | 4 | 4 | 2 |
| Example 1 | 1 | 3 | 6 |
| Example 2 | 1 | 2 | 7 |
| Example 3 | 1 | 4 | 5 |

As can be seen from the data in table 4, compared with some commercially available solid beverages, the anti-hair loss solid beverage compositions of Examples 1 to 3 provided by the present disclosure can effectively improve alopecia symptoms, and the total effective rate is more than 90%.

The present disclosure has the following beneficial effects.

(1) The anti-hair loss solid beverage according to the present disclosure takes lytodark as a main ingredient, which includes the following components: nucleoprotein peptide, coffee fruit extract, and pumpkin seed oil. Through tyrosinase activation experiments and 5-α reductase inhibition experiments, the three components in lytodark are found to have obvious synergistic effect on the activation of tyrosinase activity and inhibition of 5-α reductase activity when being used simultaneously, the effect of treating both symptoms and root causes is achieved when alopecia is treated. In addition, amino acids can be effectively supplemented, the amount of hair is increased, hair follicle aging and hair thinning are prevented, hair damage is reduced, and grey hair generation is slowed down.

(2) Compared with hormone and chemical preparation products, the anti-hair loss solid beverage provided by the present disclosure has a food function while treating alopecia, avoids side effects of the hormone and chemical preparation products, keeps hair healthy and avoids hair loss.

(3) Compared with expensive and complicated extraction processes of traditional Chinese medicinal materials, the lytodark provided by the present disclosure has a preparation process low in cost, the extraction and purification processes of components such as nucleoprotein peptide, coffee fruit extract, and pumpkin seed oil are stable, mild in condition, simple and controllable, large in subsequent industrialization potential and strong in popularization.

(4) Aiming at the deficiency of product flavor in the current market, under the premise of ensuring full play of the efficacy, the non-dairy creamer and the vegetable fat powder are used as auxiliary materials, so that the smoothness of the product taste is improved. The saturated fatty acid is provided, and the maltodextrin, the sucralose, and the essence are added, so that the sensory comfort level of the anti-hair loss product can be effectively improved, thereby improving the taste refreshing and sweet and sour, improving the edible satisfaction degree and the repeated purchase rate of consumers, and making the market competitiveness stronger.

The above-mentioned embodiments are merely intended for describing but not for limiting the technical schemes of the present disclosure. Although the present disclosure is described in detail with reference to the above-mentioned embodiments, it should be understood by those skilled in the art that, the technical schemes in each of the above-mentioned embodiments may still be modified, or some of the technical features may be equivalently replaced, while these modifications or replacements do not make the essence of the corresponding technical schemes depart from the spirit and scope of the technical schemes of each of the embodiments of the present disclosure, and should be included within the scope of the present disclosure.

1-3. (canceled)

4. A method of preparing an anti-hair loss drink mix, comprising:
preparing nucleoprotein peptide, coffee fruit extract, and pumpkin seed oil; quantitatively taking the prepared nucleoprotein peptide, coffee fruit extract, and pumpkin seed oil according to a mass ratio of 25% to 35% of the nucleoprotein peptide, 36% to 44% of the coffee fruit extract, and 26% to 38% of the pumpkin seed oil;
mixing the quantitative nucleoprotein peptide, the coffee fruit extract, and the pumpkin seed oil to obtain a tyrosinase activating substance, which is a tyrosinase activating substance;
filtering the tyrosinase activating substance, non-dairy creamer, vegetable fat powder, maltodextrin, sucralose, and essence, respectively;
quantitatively taking raw materials according to a mass ratio of 4% to 6% of the tyrosinase activating substance, 35% to 48% of the non-dairy creamer, 5.5% to 7% of the vegetable fat powder, 40% to 54% of the maltodextrin, 0.5% to 1% of the sucralose, and 0.5% to 1.5% of the essence;
mixing the quantitative maltodextrin and the non-dairy creamer to obtain a first material;
mixing the tyrosinase activating substance, the vegetable fat powder, and the first material to obtain a second material; and
mixing the quantitative sucralose, the essence, and the second material to obtain the anti-hair loss drink mix;
wherein the preparing the nucleoprotein peptide, the coffee fruit extract, and the pumpkin seed oil comprises:
preparation of the nucleoprotein peptide: cleaning, cutting, and freeze-drying deep-sea fish milt for later use; soaking the treated milt in a sodium chloride solution having a concentration in the range of 8 wt% to 15 wt% for 1 h to 2 h, followed by filtration, then washing a filter cake with deionized water and repeating for five times; adding an appropriate amount of distilled water into the soaked and filtered milt, and fully stirring, followed by adding nucleoprotease for extraction for 2 h to 4 h at a temperature of 40° C. to 60° C., the nucleoprotease having a dosage of 3500 U/g to 5500 U/g in an nucleoprotease extraction solution, and a material-liquid ratio being 1 part material to 5-10 parts liquid; heating the nucleoprotease extraction solution to 80° C. to 100° C. to inactivate the nucleoprotease, and centrifuging for 20 min at a temperature of 25° C. at 5000 rpm to obtain a supernatant purified solution; drying the purified solution at 30° C. to 40° C., and freeze-drying to obtain the desired nucleoprotein peptide;
preparation of coffee fruit extract: removing coffee beans from a fresh coffee cherry to obtain a coffee fruit; grinding the coffee fruit into powder, then dissolving the powder in an ethanol aqueous solution having a concentration of 25 wt% for extraction for 0.5 h to 2 h at a temperature of 60° C. to 70° C., the ethanol aqueous solution having a dosage of 10 mL/g to 20 mL/g in an ethanol aqueous extraction solution; concentrating the ethanol aqueous extraction solution under reduced pressure to remove a solvent, and finally drying at 50° C. to 60° C. to obtain the desired coffee fruit extract;
preparation of pumpkin seed oil: grinding fresh pumpkin seeds into powder; adding distilled water with a weight 5-8 times that of pumpkin seed powder into the pumpkin seed powder, fully stirring, followed by adding cellulase having a concentration of 0.5 wt% and pectinase having a concentration in the range of 0.1 wt%, then carrying out enzymolysis for 3 h to 5 h at a temperature of 25° C. to 40° C.; heating an enzymatic hydrolysate to 80° C. to 90° C. to inactivate the enzyme, and then centrifuging for 20 min at 5000 rpm and 25° C. to obtain an upper-layer free liquid; concentrating the upper-layer free liquid under reduced pressure, and finally drying at 30° C. to 40° C. to obtain the desired pumpkin seed oil.

5. (canceled)

6. The method for preparing the anti-hair loss drink mix according to claim 4, wherein the filtering is performed by a sieve having a mesh number of 40 meshes or 60 meshes.

7. The method for preparing the anti-hair loss drink mix according to claim 4, wherein the mixing the quantitative maltodextrin and the non-dairy creamer comprises:
adding the maltodextrin and the non-dairy creamer to a stirrer, and stirring uniformly for a first time in a closed container; the stirring for the first time has a speed of 20 r/min to 40 r/min.

8. The method for preparing the anti-hair loss drink mix according to claim 4, wherein the mixing the tyrosinase activating substance, the vegetable fat powder, and the first material comprises:
adding the tyrosinase activating substance and the vegetable fat powder to a stirrer containing the first material, and stirring uniformly for a second time in a closed container;
the stirring for the second time has a speed of 20 r/min to 40 r/min.

9. The method for preparing the anti-hair loss drink mix according to claim 4, wherein the mixing the quantitative sucralose, the essence, and the second material comprises:
adding the sucralose and the essence to a stirrer containing the second material, and
stirring uniformly for a third time in a closed container until no agglomerate is present;
the stirring for the third time has a speed of 20 r/min to 40 r/min.
